(19) United States
(12) Patent Application Publication
(10) Pub. No.: US 2008/0312249 A1
(43) Pub. Date: Dec. 18, 2008

(54) KMUP-1 CAPABLE OF TREATING HYPERTENSION

(75) Inventor: Ing-Jun CHEN, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 11/857,483

(22) Filed: Sep. 19, 2007

(30) Foreign Application Priority Data
Jun. 15, 2007 (TW) 096121950

(51) Int. Cl. A61K 31/497; A61P 43/00

(52) U.S. Cl. 514/252.16

(57) ABSTRACT

A pharmaceutical composition for treating the hypertension is provided. The pharmaceutical composition comprises a chemical compound of 7-[2-[4-(2-Chlorobenzene)piperazinyl]ethyl]-1,3-dimethyl xanthine and one of a pharmaceutical acceptable salt thereof and a solvate thereof, wherein the chemical compound treats the hypertension by cGMP-dependent inhibition on Rho kinase.

### FIELD OF THE INVENTION

The present invention relates to a theophylline-based compound capable of enhancing the production of cGMP, and more particularly to a compound of 7-[2-[4-(2-Chlorobenzene)piperazinyl]ethyl]-1,3-dimethyl xanthine capable of treating a hypertension by cGMP-dependent inhibition on Rho kinase.

### BACKGROUND OF THE INVENTION

U.S. Pat. No. 6,979,687 disclosed a serial of theothylline-based compounds, KMUP-1 and KUMP-2, having a minimum inhibition on phosphodiester (PDE), and capable of activating the soluble guanynyl cyclase (sGC). The inhibition on PDE enhances the concentration of cGMP and the activation on sGC promotes the production of cGMP. cGMP molecule modulates the regulation of the NO-releasing relevant proteins relaxes the blood vessels. Therefore, it has been proven in the mentioned patent that KMUP-1 contributes to the relaxation of the blood vessels of the corpus cavernosal in the penis.

In the previous studies, it has been demonstrated that the up-regulation of cGMP could (1) phosphorelate RhoA protein, which deactivates RhoA kinase; (2) phosphorelate IRAG so that the regulation of IP3/IRAG on calcium ions in the sarcoplasmic reticulum is inhibited; and (3) phosphorylate potassium ion channels by Protein Kinase G (PKG).^{6, 7, 21}

cGMP and Rho kinase (ROCK) play crucial roles in the regulation on pulmonary arterial contractility. Untoward vascular contraction and resistance are remained to be solved in associated pulmonary artery hypertension (PAHT).¹ ROCK-mediated Ca²⁺sensitization plays the central role in mediating the increased vasoreactivity, sustained vasoconstriction and hypertension.^{1˜4} Production of cGMP may be impaired by inactivation of eNOS due to endothelium dysfunction.⁸⁻¹⁰

It has been known that (1) cGMP-dependent protein kinase signaling pathway inhibits RhoA-induced Ca²⁺-sensitization in vascular smooth muscle contraction;¹¹(2) action downstream of cGMP can reverse PKC-mediated Ca²⁺-sensitization; (3) cGMP-dependent protein kinase is regulated by Rho protein.¹²

The commercial U46619 could induce an animal model of PAHT, displaying sustained increase of vascular contractility and resistance in pulmonary artery. However, it is still unknown whether the cGMP enhancer, KMUP-1, can inhibit PAHT by K⁺-channel opening and actions on co-localized vascular eNOS/sGC/PDE5A and PKCα/ROCK in the presence of U46619.

It has been mentioned that there are two kinds of treatments for PAHT; one is to enhance the production of cGMP either with the PDE5 inhibitor, sildenafil, or with the sGC activator, Bay-41-2272; the other is to inhibit ROCK with Y27632.^{13˜16} The commercial drugs, Sildenafil, BAY-41-2272 and Y27632, have encouraged us to search for a cGMP-dependent type ROCK inhibitor, KMUP-1, to inhibit PAHT.

From the above description, it is known whether KMUP-1 involves in the inhibition of PAHT has become a major problem waited to be solved. In order to overcome the drawbacks in the prior art, another pharmaceutical activity of KMUP-1 is provided. The particular design in the present invention not only solves the problems described above, but also is easy to be implemented. Thus, the invention has the utility for the industry.

### SUMMARY OF THE INVENTION

In the present invention, we first show that KMUP-1, a cGMP-dependent type ROCK inhibitor, inhibits U46619-induced PAHT and vascular contraction and enhances pulmonary arterial expression of endothelium nitric oxide synthase (eNOS), soluble guanylate cyclase (sGC), protein kinase G (PKG) and opposing reduction of ROCK, phosphodiesterase 5A (PDE5A) and translocation of protein kinase C (PKC_{α}) in isolated intact rat pulmonary artery.

In accordance with one aspect of the present invention, a method for treating a hypertension is provided. The method comprises a step of administering to a mammal a therapeutically effective amount of a compound of 7-[2-[4-(2-Chlorobenzene)piperazinyl]ethyl]-1,3-dimethyl xanthine.

Preferably, the administration is one selected from a group consisting of an oral injection, an intraperitoneal injection and an intravenous injection.

Preferably, the hypertension is a pulmonary hypertension.

Preferably, the hypertension is a spontaneous hypertension.

Preferably, the compound causes a cGMP-dependent inhibition on Rho kinase.

Preferably, the method further comprises a step pf administering the compound with the pharmaceutically effective carrier thereof.

The above aspects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a schematic diagram showing the effects of oral KMUP-1 on the mean pulmonary arterial pressure (MPAP) of U46619-induced rats. KMUP-1 was administered by dosages 15, 20, 25 mg/kg. U46619 was intravenously infused at 2.5 μg/min/kg for 20 min. Values are means±S.E., n=6, *, P<0.05; **, P<0.01 versus control and U46619-treated vessels by Dunnett multiple range test;

FIG. 2 is a schematic diagram showing the effects of intraperitoneal KMUP-1 on the MPAP of U46619-induced rats. Values are means±S.E., n=6, *indicated P<0.05, **indicated P<0.01 compared with the U46619-treated group by Dunnett multiple range test;

FIG. 3 is a schematic diagram showing the effects of intravenous KMUP-1 on MPAP of U46619-induced rats. Values are means±S.E., n=6, *indicated P<0.05, **indicated P<0.01 compared with the U46619-treated group by Dunnett multiple range test;

FIG. 4 is a schematic diagram showing the effects of KMUP-1 and the commercial drugs, Milrinone, Zaprinast and Urapidil on the MPAP. KMUP-1 (1.0 μg kg⁻¹ min⁻¹), KMUP-3 (0.75 μg kg⁻¹ min⁻¹), Milrinone (1 μg kg⁻¹ min⁻¹), Zaprinast (1 μg kg⁻¹ min⁻¹), and Urapidil (1 μg kg⁻¹ min⁻¹) were infused in rats. Values are means±S.E., n=6, *, P<0.05; **, P<0.01 versus control and treated vessels by Dunnett multiple range test;

FIG. 5 is a schematic diagram showing the effects of KMUP-1 and the commercial drugs, Milrinone, Zaprinast and Urapidil, on the MPAP of rats before U46619 is infused therein. KMUP-1 (2.0 μg kg⁻¹ min⁻¹), KMUP-3 (0.75 μg kg⁻¹ min⁻¹), Milrinone (1.0 μg kg⁻¹ min⁻¹), Zaprinast (1.0 μg kg⁻¹ min⁻¹) and Urapidil (1.0 μg kg⁻¹ min⁻¹) were infused in rats. Values are means±S.E., n=6, *, P<0.05; **, P<0.01 versus control and treatment vessels before U46619-infusion by Dunnett multiple range test;

FIG. 6 is a schematic diagram showing the effects of KMUP-1 and the commercial drugs, Milrinone, Zaprinast and Urapidil, on the heart rate of rats. KMUP-1 (1.0 μg kg⁻¹ min⁻¹), KMUP-3 (0.75 μg kg⁻¹ min⁻¹), Milrinone (1.0 μg kg⁻¹ min⁻¹), Zaprinast (1.0 μg kg⁻¹ min⁻¹) and Urapidil (1.0 μg, kg⁻¹, min⁻¹) were infused in rats. Values are means±S.E., n=6, *, P<0.05; **, P<0.01 versus control and treated vessels by Dunnett multiple range test;

FIG. 7 is a schematic diagram showing anti-hypertension activities of KMUP-1 on SHR and WKY rats. KMUP-1 (10 and 30 mg/kg) was orally administered in SHR (S) and WKY (W) rats, aging at 8 weeks. S: 0 mg control; S1: 10 mg/kg; S2: 30 mg/kg; W: 0 mg control; W1: 10 mg/kg; W2: 30 mg/kg. Values are means±S.E., n=6, *, P<0.05; **, P<0.01 versus control and treatment group by Dunnett multiple range test;

FIG. 8 is a schematic diagram showing the relaxation effects of KMUP-1 on phenylephrine-induced contraction of rat pulmonary artery ring. KMUP-1, KMUP-3, Milrinone, Sildenafil, Zapinast and Urapidil at 100 μM were applied in organ bath. Values are means±S.E., n=6, *, P<0.05; **, P<0.01 versus control and treatment vessels by Dunnett multiple range test;

FIG. 9 is a schematic diagram showing the relaxation effects of KMUP-1 on U46619-induced contraction of rat pulmonary artery ring. KMUP-1, KMUP-3, Milrinone, Sildenafil, Zapinast and Urapidil at 100 μM were applied in organ bath. Values are means±S.E., n=6, *, P<0.05; **, P<0.01 versus control and treatment vessels by Dunnett multiple range test;

FIG. 10 is a schematic diagram showing the relaxation effects of KMUP-1 on phenylephrine-induced contraction of rat pulmonary artery ring, wherein PMA, ODQ, L-NAME and SQ22536 were applied before application of KMUP-1 (100 μM) in organ bath. Values are means±S.E., n=6, *, P<0.05; **, P<0.01 versus control and U46619-treated vessels by Dunnett multiple range test;

FIG. 11 is a schematic diagram showing the effects of KMUP-1 on U46619-induced interaction between ROCK and eNOS expression in rat pulmonary arteries. Isolated blood vessels were incubated with a series concentration of KMUP-1 (0.1, 1.0, 10 μM) for 60 min and then added with or without U46619 (0.5 μM) for another 60 min. Values are means±S.E., n=6, *, P<0.05; **, P<0.01 versus U46619-treated vessels by Dunnett multiple range test;

FIG. 12 is a schematic diagram showing the effects of KMUP-1 on U46619-induced interaction between eNOS, sGC_{α} and sGC_{β} expressions in rat pulmonary arteries. Isolated blood vessels were incubated with KMUP-1 (10 μM); L-NAME (10 μM); ODQ (10 μM) for 60 min and then added with or without U46619 (0.5 μM) for another 60 min. Values are means±S.E., n=6, *, P<0.05; **, P<0.01 versus U46619-treated vessels by Dunnett multiple range test;

FIGS. 13(A) and (B) are schematic diagrams showing the effects of KMUP-1 on the expression of ROCK and PKG in rat pulmonary artery ring in the presence and absence of cGMP-inhibitor (RP-8p-CPT-cGMP). Isolated blood vessels were incubated with KMUP-1 (10 μM) and Y27632 (100 μM) for 60 mins and then added with RP-8p-CPT-cGMP (100 mM) for another 60 min. Values are means±S.E., n=6, *, P<0.05; **, P<0.01 versus control and U46619-treated vessels by Dunnett multiple range test;

FIG. 14 is a schematic diagram showing the effect of KMUP-1 on U46619-induced PKCα translocation in rat pulmonary artery rings. Isolated blood vessels were incubated with KMUP-1 (100 μM) for 60 min and then added with or without U46619 (0.5 μM) for another 60 min. Values are means±S.E., n=6, *, P<0.05; **, P<0.01 versus control and U46619-treated vessels, respectively, by Dunnett multiple range test; and

FIG. 15 is a schematic diagram showing the effects of KMUP-1 on the expression of PKC_{α} on the cytosol and membrane in the presence and absence of U46619. Values are means±S.E., n=6, *, P<0.05; **, P<0.01 versus control and treated vessels by Dunnett multiple range test.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention provides a chemical compound, KMUP-1, having a pharmaceutical activity of anti-hypertension. The detailed description for the pharmaceutical experimental results of KMUP-1 is provided as below.

Pharmaceutical Trials

1. The Preparation for the Present Chemical Compound

The preparation of KMUP-1 has been disclosed in U.S. Pat. No. 6,969,687, and thus it will not be mentioned again in the present invention.

2. Testing of Blood Pressure

Pulmonary artery pressure was induced with U46619 (2.5 μg kg⁻¹ min⁻¹×20 min). Mean Pulmonary Artery Pressure (MPAP) was recorded from pulmonary artery of open-chest rats. Oral, intraperitoneal and intravenous KMUP-1 were administered 30, 30 and 20 min, respectively, before U46619 infusion. Tail artery blood pressure of rat (SBP) was measured from 8 weeks old SHR and WKY, treated with KMUP-1 and vehicle for 4 weeks. Increased blood pressure=Blood pressure at week 9˜12—Initial blood pressure at week 8.

3. Testing of Pulmonary Artery Tension

The arterial rings (2˜3 mm) were isometricly connected to a force transducer and amplifier as previously.⁶ Relaxation was estimated as following: Relaxation %=relaxation by KMUP-1—relaxation by solvent/relaxation by KMUP-1.

4. Western Blot Analysis

Pulmonary arterial rings were incubated with U46619 (0.5 μM) or PE (1.0 μM) for 60 min and then treated with KMUP-1 for 60 min. Proteins expression of arterial rings were analyzed using mouse monoclonal antibody.¹⁸ Pretreatment with inhibitors and KMUP-1 were 60 min before application of U46619.

5. BKCa and Ca²⁺ Currents

Smooth muscle cells from rat pulmonary artery were enzymatically isolated and incubated in 0.5 mg/ml collagenase IA, 0.6 mg/ml papain and 0.2 mg/ml dithioerythritol for 45 min. Whole cell BKCa and Ca2+(IBa) current were measured using the conventional patch-clamp configuration on cells.²³

6. Calcium Mobilization and [Ca²⁺](i)

Smooth muscle cells from rat pulmonary arteries were loaded with Fura-2/AM to permit measurement of [Ca²⁺](i) changes in single cells by a spectroflurophotometer (Shimadzu, RF-5301PC, Japan).

Vascular eNOS and sGC are involved in a multimeric complex.¹⁷ Likewise, KMUP-1, Bay-41-2271 and sildenafil, predominately mimic the action of cGMP by regulating a eNOS/sGC/PDE mixed functional enzyme system.¹⁸ In contrast, ROCK, involved in the G protein-dependent Ca2+-sensitization and agonist-activated Ca2+ entry, can suppress co-localized eNOS.^{19,20}

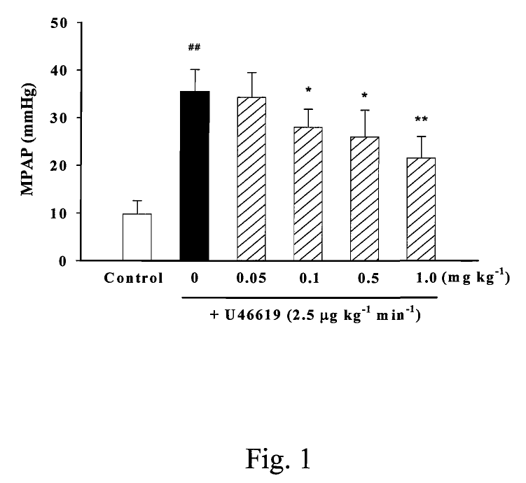

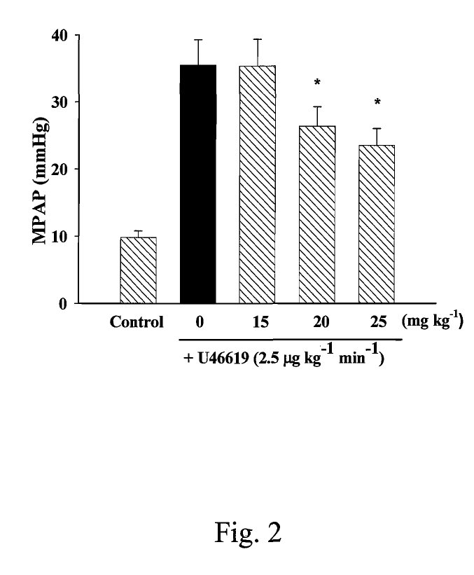

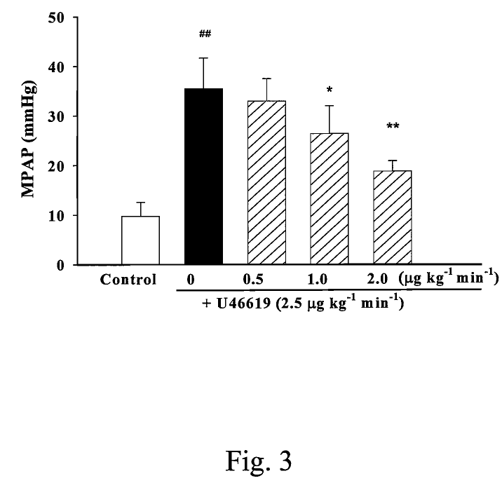

Please refer to FIGS. 1˜3, which show the effects of KMUP-1 on the MPAP of U46619-induced rats by an oral injection, an intraperitoneal injection and an intravenous injection. Oral KMUP-1 was administered by dosages 15, 20, 30 mg/kg, intravenous KMUP-1 was administered by dosage 0.5, 1.0 and 2.0 mg/kg and intraperitoneal KMUP-1 was administrated by dosages 0.05, 0.1 and 1.0 mg/kg. The results in FIGS. 1 to 3 indicate that the inhibition of KMUP-1 on U46619-induced MPAP correlates with the administered dosages.

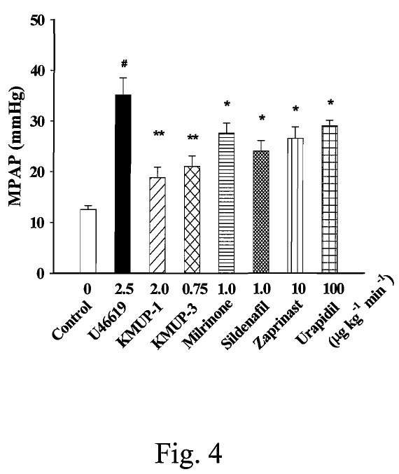

Please refer to FIG. 4, which shows of the effect of KMUP-1 on the MPAP of the U46619-induced rats as compared to those with the administration of the commercial drugs, Milrinone, ildenafil, Zaprinast and Urapidil. The result of FIG. 4 indicates that the commercial drugs, Milrinone, ildenafil, Zaprinast and Urapidil, are able to decrease the MPAP of the U46619-induced rats, wherein KMUP-1 of the present invention has the most significant inhibition thereover.

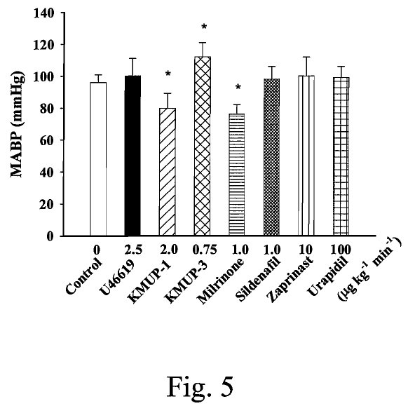

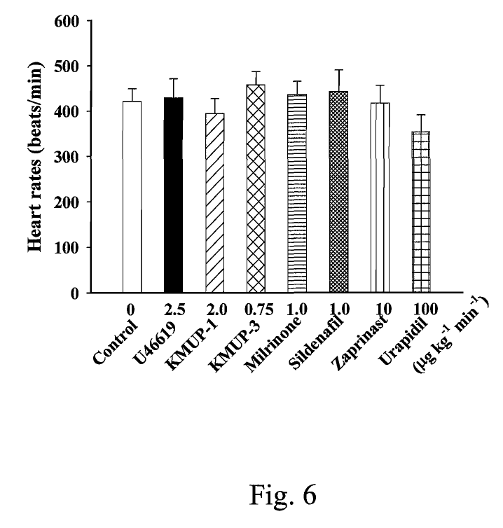

Please refer to FIG. 5, which shows the effects of KMUP-1 and the commercial drugs, Milinone, Zaprinast and Urapidil on the MPAP before U46619 is administered thereto. Please refer to FIG. 6, which shows the effects of KMUP-1 and the commercial drugs, Milirone, Zaprinast and Urapidil on the rats' heart rate. It is known from the results of FIGS. 5 and 6 that KMUP-1 and the commercial drugs, Milrinone, Zaprinast and Urapidil have no significant effect on the systematic MPAP and the heart rate.

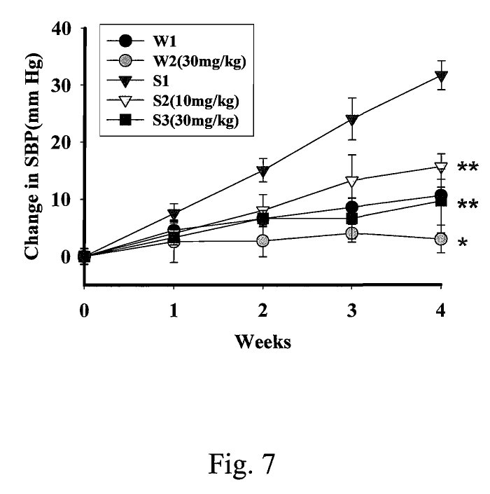

Please refer to FIG. 7, which shows anti-hypertension activity of KMUP-1 on spontaneous hypertension rats (SHR) and non-genomic disease type WKY rats (WKY). The respective amounts, 10 mg/kg and 30 mg/kg, of KMUP-1 are orally administered to SHR (S) and WKY (W) rats with eight weeks old. SI represents a control group without the injection of KMUP-1. It is known from the result that KMUP-1 of the present invention has a significant effect on inhibiting the blood pressure of SHR, whereas it has a little effect on inhibiting the blood pressure of WKY.

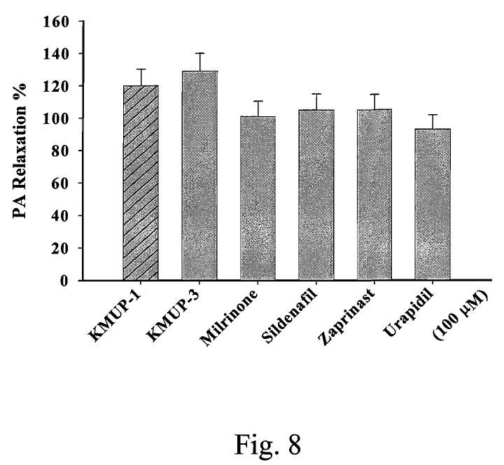

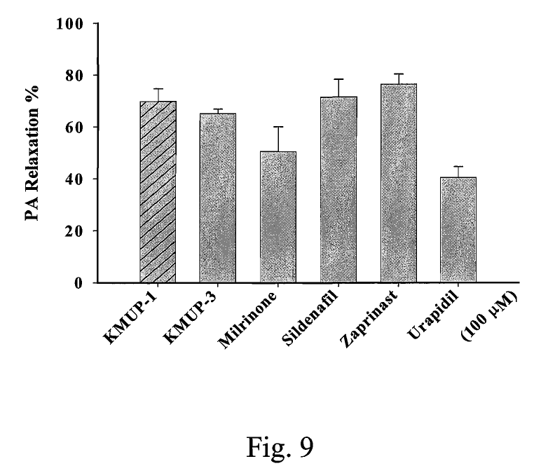

Please refer to FIGS. 8 and 9, which show the effects of KMUP-1 on the contractility and relaxation of the rats' pulmonary arterial rings induced by phenylephrine and U46619, respectively. The amount of the injection of KMUP-1, KMUP-3, Milrinone, Sildenafil, Zapinast and Urapidil is 100 μM. It is known from the result of FIG. 8 that KMUP-1 can inhibit the contractility of the isolated pulmonary arterial rings induced by phenylephrine and U46619, respectively. Therefore, KMUP-1 of the present invention can inhibit Ca²⁺-sensitization and Ca²⁻ entry in vascular smooth muscle.^{21, 5}

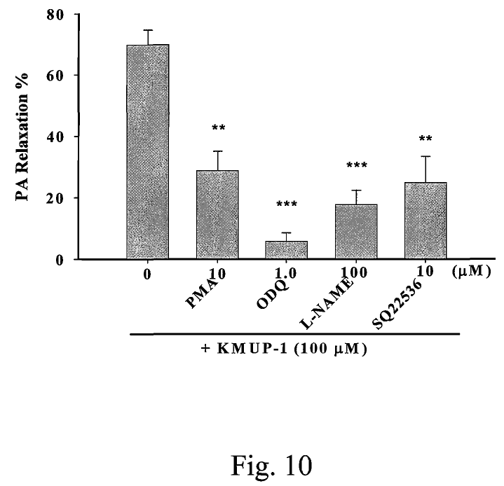

Please refer to FIG. 10, which shows the effect of KMUP-1 on the relaxation of the pulmonary arterial rings induced by phenylephrine. PMA, ODQ, L-NAME and SQ22536 were applied before application of KMUP-1 (100 μM) in organ bath. It is known that the effect of KMUP-1 on the contractility of the rats' pulmonary artery rings induced by U46619 will be reduced by application of eNOS inhibitor L-NAME (100 μM), sGC inhibitor ODQ (1 μM), cAMP inhibitor SQ22536 (10 μM) and PKC activator Phorbol 12-myristate, 13-acetate (PMA) (10 μM). Therefore, these evidences indicated the partial involvement of eNOS/sGC in vascular relaxation of pulmonary artery.

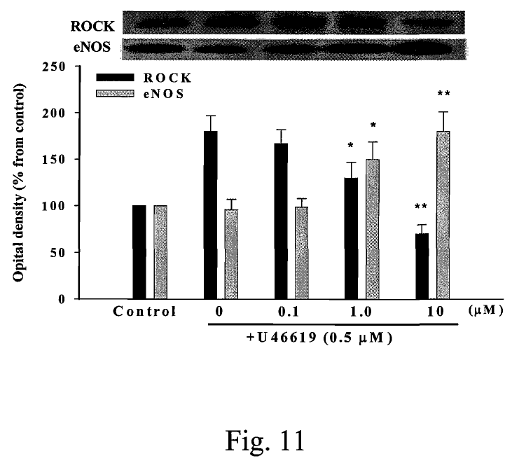

Please refer to FIG. 11, which shows the effect of KMUP-1 on the expression of ROCK and eNOS in the pulmonary artery rings induced by U46619. The isolated blood vessels are incubated for 60 mins in the treatment of the respective amounts, 0.1, 1.0 and 10 μM, of KMUP-1. The result indicates that the expression of ROCK has a positive correlation with the dosage of KMUP-1, whereas the expression of eNOS has a negative correlation with the dosage of KMUP-1.

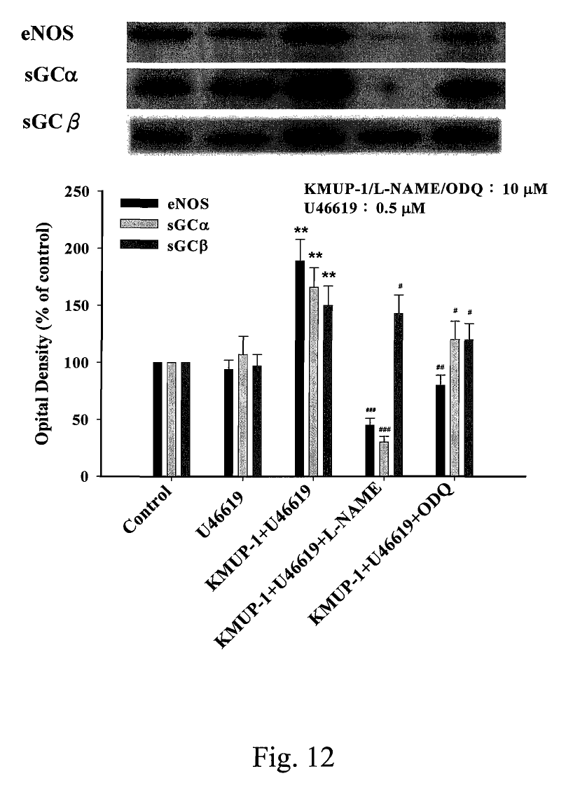

Please refer to FIG. 12, which show the effect of KMUP-1 on the expression of eNOS, sGC_{α} and sGC_{β} in the pulmonary artery rings induced by U46619. One sample groups of the isolated blood vessels are treated with U46619 for 60 mins. Another sample group of the isolated blood vessels are treated with U46619 for 60 mins after the treatment of KMUP-1 for 60 mins. The result indicates that the expression of eNOS, sGC_{α} and sGC_{β} treated only by U46619 is similar to that of the control group, whereas the expression of eNOS, sGC_{α} and sGC_{β} treated by KMUP-1 and U46619 is significantly raised. Followed by the treatment of L-NAME and ODQ, the expression of eNOS is significantly reduced.

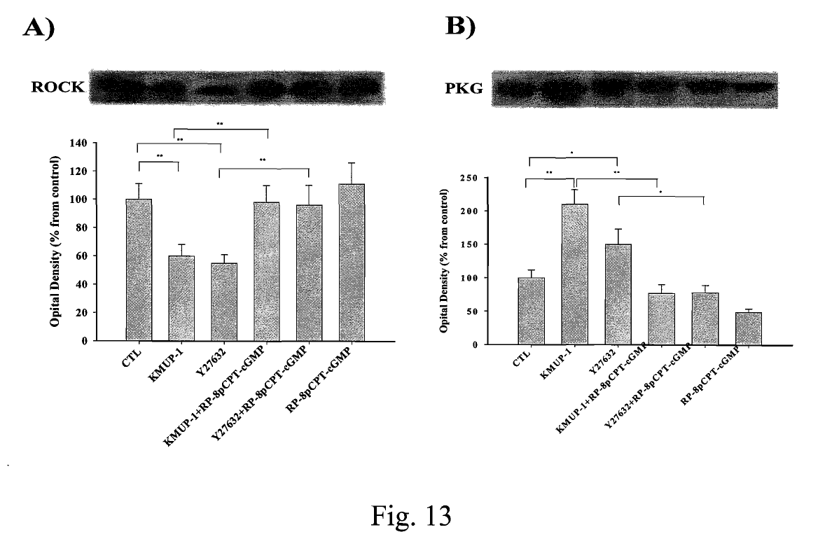

Please refer to FIGS. 13(A) and 13(B), which show the effect of KMUP-1 and Y27632 on the expression of ROCK (A) and PKG (B). The isolated blood vessels are cultured with the respective KMUP-1 (100 μM), Y27632 (100 μM) and RP-8pCPT-cGMP (100 μM). FIG. 13(A) indicates that the effect of KMUP-1 and Y27632 on the expression of ROCK in the presence and absence of RP-8pCPT-cGMP and FIG. 13(b) indicates that the effect of KMUP-1 and Y27632 on the expression of PKG in the presence and absence of RP-8pCPT-cGMP. The result of FIG. 13(A) indicates that KMUP-1 has a similar inhibition on the expression of ROCK to Y27632, and the result of FIG. 13(B) indicates KMUP-1 has a greater activation on the expression of PKG than Y27632. However, the effect of KMUP-1 on reducing the expression of ROCK and activating the expression of PKG will be recovered by the cGMP-inhibitor. As the above, the action of KMUP-1 depends on cGMP since ROCK primarily regulates downstream of eNOS. Namely, the effect of KMUP-1 on inhibiting the expression of ROCK is achieved by enhancing the expression of eNOS. In contrast, the direct activation of KMUP-1 on eNOS attributes to the inhibition of ROCK.

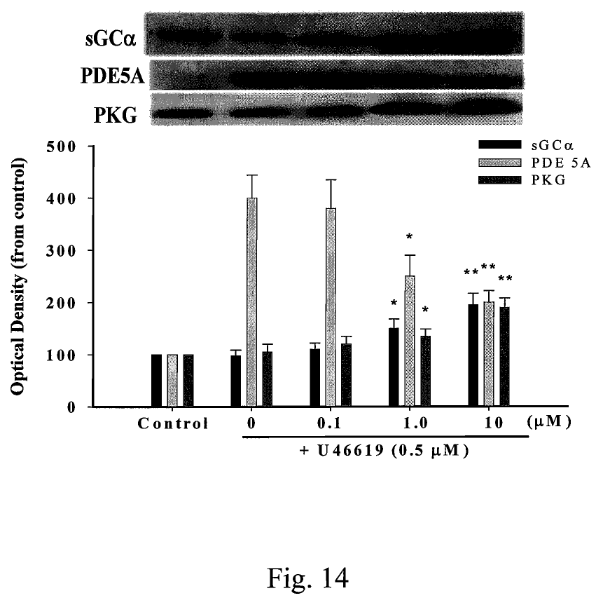

Please refer to FIG. 14, which shows the effect of KMUP-1 on the translocation of PKC_{α} in the pulmonary artery rings of rats induced by U46619. The isolated blood vessels are cultured with KMUP-1 for 60 mins, followed by adding U46619 for 60 mins, and the expression of sGC_{α}, sGC_{β} and PKG thereof are measured. The result of FIG. 14 indicates that the expression of sGCa and PKG has a positive correlation with the dosage of KMUP-1 and the expression of PDE5A has a negative correlation with the dosage of KMUP-1. Even in the pretreatment of L-NAME and ODQ, KMUP-1 still inhibits the expression of ROCK (data not shown). Therefore, the cGMP-dependent ability of KMUP-1 not only through sGC activation but also PDE5A inhibition, which is associated with the presence of U46619. Furthermore, cGMP inhibitor, Rp-8-CPT-cGMP, can blunt KMUP-1-induced reduction of ROCK, the increase of PKG and PKC_{α} translocation, further indicating the cGMP-dependent and Rho kinase inhibition properties of KMUP-1.

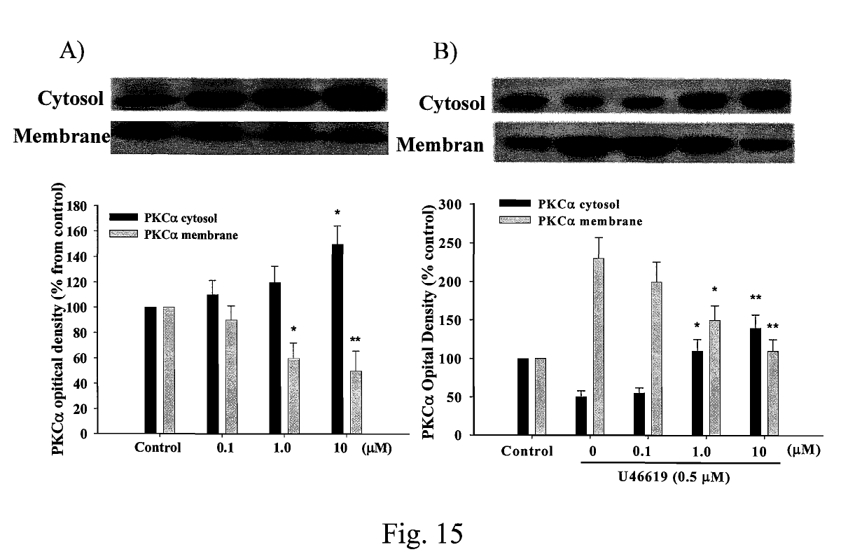

Please refer to FIG. 15, which shows the effect of KMUP-1 with different concentrations on the expression of PKC_{α} on the cytosol and membrane in the presence and absence of U46619. The result of FIG. 15 indicates that the expression of PKC_{α} on the membrane reduces with the increased dosage of KMUP-1.

As mentioned in the above, the present invention indicates that KMUP-1 involves in the multi-enzyme composite system of the upstream of cGMP, inhibits the expression of ROCK and further involves in the translocation of PKC_{α} in the isolated intact pulmonary artery. Therefore, KMUP-1 provided by the present invention is capable of inhibiting PAHT and artery hypertension by means of activating the synthesis of cGMP and reducing the expression of ROCK.

Accordingly, the present invention can effectively solve the problems and drawbacks in the prior art, and thus it fits the demand of the industry and is industrially valuable.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

1. A method for treating a hypertension, comprising a step of administering to a mammal a therapeutically effective amount of a compound of 7-[2-[4-(2-Chlorobenzene)piperazinyl]ethyl]-1,3-dimethyl xanthine.

2. A method as claimed in claim 1, wherein the administration is one selected from a group consisting of an oral injection, an intraperitoneal injection and an intravenous injection.

3. A method as claimed in claim 1, wherein the hypertension is a pulmonary hypertension.

4. A method as claimed in claim 1, wherein the hypertension is a spontaneous hypertension.

5. A method as claimed in claim 1, wherein the compound causes a cGMP-dependent inhibition on Rho kinase.

6. A method as claimed in claim 1, further comprising a step of administering the compound with the pharmaceutically effective carrier thereof.

7. A method for treating a hypertension, comprising a step of administering to a mammal a therapeutically effective amount of compounds of 7-[2- [4-(2-fluorobenzene)piperazinyl]ethyl]-1,3-dimethyl xanthine and 7-[2-[4-(2-trifluoromethylbenzene)piperazinyl]ethyl]-1,3-dimethyl xanthine.
